(19) United States
(12) Patent Application Publication
(10) Pub. No.: US 2023/0043428 A1
(43) Pub. Date: Feb. 9, 2023

(54) COMPOSITIONS AND METHODS FOR USE OF CANNABINOIDS FOR NEUROPROTECTION

(71) Applicant: INMED PHARMACEUTICALS INC., Vancouver (CA)

(72) Inventors: Eric HSU, Vancouver (CA); Ujendra KUMAR, Richmond (CA); Rishi Kumar SOMVANSHI, Vancouver (CA); Shenglong ZOU, Vancouver (CA)

(21) Appl. No.: 17/606,003

(22) PCT Filed: Apr. 24, 2020
(86) PCT No.: PCT/CA2020/050547

§ 371 Date: Oct. 22, 2021

Related U.S. Application Data
(60) Provisional application No. 62/838,216, filed on Apr. 24, 2019.

(51) Int. Cl. A61K 31/352; A61K 9/00; A61P 25/28; A61P 27/06

(52) U.S. Cl. CPC A61K 31/352; A61K 9/0048; A61P 25/28; A61P 27/06

(57) ABSTRACT

Provided herein are methods and compositions for neuroprotection. The neuroprotective composition can be or include cannabinol or a derivative thereof. The neuroprotective composition can be used in the treatment of a neurodegenerative disease. The neuroprotective composition can be used to protect retinal neurons from degeneration in a subject in need thereof, such as for treatment of glaucoma.

### CROSS-REFERENCES TO RELATED APPLICATIONS

This application claims the benefit of priority to U.S. Provisional Application No. 62/838,216, filed Apr. 24, 2019, the contents of which are hereby incorporated by reference in the entirety and for all purposes.

### BACKGROUND OF THE INVENTION

Neurodegeneration is a phenomenon underlying a wide array of different diseases of the central and peripheral nervous system. Neurodegeneration includes neuronal atrophy, axonal degeneration (e.g., Wallerian and/or Wallerian-like degeneration), and induction of necrotic or programmed mechanisms of cell death. Different types of programmed cell death such as apoptosis, autophagy, pyroptosis, and oncosis have all been demonstrated in neurons. Stimuli such as physical injury, oxidative stress, excitotoxicity, mitochondrial dysfunction, inflammation, iron accumulation, and protein aggregation have all be shown to contribute to mechanisms of neurodegeneration.

Glaucoma is a form of optic neurodegeneration characterized by progressive degeneration of the retinal ganglion cells, which are cells of the central nervous system, positioned such that the cell body is located within the retina, and the axon is located in the optic nerve. Degeneration of these neurons is associated with a progressive loss of vision and a characteristic morphology of the optical disc referred to as “cupping”.

The etiology of glaucoma are poorly understood and the factors contributing to its progression have not yet been fully characterized. Glaucoma affects more than 70 million people worldwide, 10% of whom lose their vision because of this disease. Glaucoma has no symptoms in the early and intermediate stages, so the number of people suffering from glaucoma may be much higher than that of people diagnosed with this disease. In fact, several surveys have shown that less than 50% of people diagnosed with glaucoma were aware of being affected by this disease. Glaucoma can be classified into 2 broad categories: open-angle glaucoma and closed-angle glaucoma. In the United States, more than 80% of glaucoma cases are open-angle cases.

Glaucoma may be primary, i.e. without a well-defined cause, or secondary, resulting from trauma, glucocorticoids, pigment dispersion or pseudo-exfoliation syndrome. Although, as indicated, the mechanism of pathogenesis of glaucoma is not fully understood, an increase in the intraocular pressure is known to be associated with the neurodegeneration of retinal ganglion cells. Intraocular pressure is determined by the balance between aqueous humor secretion by ciliary bodies and its drainage via trabecular and uveoscleral outflow.

Patients with open-angle glaucoma show a reduction in the outflow of aqueous humor due to partial obstruction of the trabecular and uveoscleral ducts. Intraocular pressure can cause stress and mechanical strain on the rear structures of the eye, particularly the lamina cribrosa and adjacent tissues. Stress and mechanical strain-induced by increased intraocular pressure-can lead to compression, deformation and re-modelling of the lamina cribrosa, with consequent impairment of axonal transport of trophic factors essential for retinal ganglion cells. The death of retinal ganglion cells has been shown to be able to induce neurodegeneration of surrounding neurons, leading to secondary and trans-synaptic neuronal damage, which may be of great importance in the progression of the disease.

The increase in intraocular pressure is not the only risk factor, since individuals with high intraocular pressure may not develop the disease, while in some cases the hypotensive therapy alone proves ineffective in slowing down or stopping the progression of the disease. Alterations of the microcirculation and the immune system, excitotoxicity and oxidative stress may also contribute to the development of optic neurodegeneration, both in the presence and in the absence of high intraocular pressure values.

One of the few methods currently known and effective in the treatment of neurodegeneration in glaucoma is the reduction of intraocular pressure. Numerous multicentre studies have shown the benefit resulting from the reduction of intraocular pressure in the prevention of the onset and in the slowing down of the progression of this disease. However, reduction of intraocular pressure is not always effective. Moreover, even in subjects in which reduction of intraocular pressure shows efficacy, disease progression may not be halted and existing damage cannot be reversed.

Other forms of eye diseases associated with neural degeneration include age-related macular degeneration (AMD), diabetic retinopathy, and retinitis pigmentosa. Age-related macular degeneration (AMD) affects about 14-24% of the people aged 65 to 74 and about 35% of the people over 75 around the world, and results in vision impairment or loss in the center of the visual field (the macula) because of damage to the retina and/or associated neurons. It is a major cause of vision loss and potentially blindness in people over 50 years of age. The two principal forms of AMD are atrophic (non-exudative or “dry”) AMD and neovascular (exudative or “wet”) AMD. Atrophic AMD is characterized by geographic atrophy (GA) at the center of the macula in the advanced stage of AMD, and vision can slowly deteriorate over many years due to loss of photoreceptors and development of GA. Neovascular AMD is a more severe form of AMD and is characterized by neovascularization (e.g., choroidal neovascularization) in the advanced stage of AMD, which can rapidly lead to blindness. Neovascular AMD affects more than 30 million patients worldwide and is a leading cause of vision loss in people aged 60 years or older—if untreated, patients are likely to lose central vision in the affected eye within 24 months of disease onset. About 90% of AMD patients have the dry form, and about 10% develop neovascular AMD.

Diabetic retinopathy is a complication of diabetes, which is caused by hyperglycemia-induced incompetence of the vascular walls, resulting in microvascular retinal changes, such as dysfunction of blood-retinal barrier and hyperpermeability of capillary circulation. Subsequently, both capillary- and neuro-degeneration result in severe vision defects. Diabetic retinopathy is the leading cause of blindness in patients with diabetes.

Conventional methods for relieving symptoms of diabetic retinopathy include laser surgery, vitrectomy and intraocular injection of corticosteroids. However, all of the conventional methods belong to invasive treatments but cannot completely cure diabetic retinopathy. Therefore, patients with diabetic retinopathy have to monitor blood glucose level to adapt to maintain normal blood glucose level (euglycemia) all the time. Furthermore, intraocular injection of corticosteroids can also lead to side effects such as steroid-induced disorders. In light of this, it is necessary to improve the conventional method for relieving symptoms of diabetic retinopathy.

Retinitis pigmentosa is a slowly progressive, bilateral degeneration of the retina, retinal neurons, and retinal pigment epithelium caused by various genetic mutations. Symptoms include night blindness and loss of peripheral vision.

Neuroprotection is an effect that can provide salvage, or recovery of the nervous system, its cells, structure, and/or function, or resistance to neurodegenerative stimuli. Neuroprotective compositions may find use in treating a variety of diseases that cause or result in neurodegeneration, such as glaucoma, or mitigating their symptoms. Despite significant advances in understanding the underlying mechanisms of neurodegeneration, there remains a need in the art for improved methods and compositions for neuroprotection.

Cannabinoids, and derivatives thereof, have several properties with therapeutic potential. Activation or blocking of CB1 and/or CB2 receptors with a cannabinoid can regulate downstream signaling and metabolic pathways and subsequently influence synaptic transmission, including transmission of pain and other sensory signals in the periphery, immune response, and inflammation. Thus, there is an interest in the use of natural or synthetic cannabinoids for therapeutic purposes. However, despite anecdotal reports of cannabinoid therapeutic effects, many cannabinoids and their derivatives have been demonstrated to exhibit no detectable neuroprotective effect at physiological concentrations. Moreover, some cannabinoids and their derivatives have been shown to contribute to excitotoxicity at physiological concentrations.

### SUMMARY OF THE INVENTION

Described herein are neuroprotective compositions and formulations, and methods of their making and use. A neuroprotective composition, or a formulation containing a neuroprotective composition, can be contacted with a neuron, thereby providing a neuroprotective effect. In certain embodiments, the contacting is performed by administering the neuroprotective composition, or a formulation containing the composition, to a subject in need thereof. The neuroprotective compositions, formulations, and related methods are useful in treating a wide variety of neurodegenerative diseases. In certain embodiments, neuroprotective compositions are provided for use in inducing a neuroprotective effect in retinal neurons. For example, a neuroprotective composition can be locally or systemically administered to a subject to induce a neuroprotective effect in retinal neurons, e.g., to treat an optical neurodegenerative disease such as glaucoma or inhibit neurodegeneration associated with diabetic retinopathy, AMD, and/or retinitis pigmentosa.

In one aspect, the present invention provides a method of protecting a neuron from neurodegeneration, the method comprising contacting the neuron with a composition comprising cannabinol, or a derivative thereof, in an amount sufficient to inhibit neurodegeneration. In some embodiments, the contacting is in vitro. In some embodiments, the contacting is in vivo. In some embodiments, the contacting comprises administering the composition to a subject in need thereof. In some embodiments, the neuron is a retinal neuron (e.g., retinal ganglion).

In some embodiments, the contacting comprises topically administering the composition to a subject in need thereof. For example, administering the composition to a subject suffering from neurodegeneration, e.g., neurodegeneration of the eye. In some cases, the contacting comprises administering the composition to a subject suffering from a neurodegenerative disease, e.g., a neurodegenerative disease of the eye. In some cases, the contacting comprises administering the composition to a subject suffering from glaucoma. In some cases, the contacting comprises administering the composition to a subject diagnosed with glaucoma. In some embodiments, the method comprises simultaneously or sequentially administering an additional active agent for treatment of glaucoma.

In some cases, the contacting comprises administering the composition to a subject suffering from AMD. In some cases, the contacting comprises administering the composition to a subject diagnosed with AMD. In some embodiments, the method comprises simultaneously or sequentially administering an additional active agent for treatment of AMD.

In some cases, the contacting comprises administering the composition to a subject suffering from diabetic retinopathy. In some cases, the contacting comprises administering the composition to a subject diagnosed with diabetic retinopathy. In some embodiments, the method comprises simultaneously or sequentially administering an additional active agent for treatment of diabetic retinopathy.

In some cases, the contacting comprises administering the composition to a subject suffering from retinitis pigmentosa. In some cases, the contacting comprises administering the composition to a subject diagnosed with retinitis pigmentosa. In some embodiments, the method comprises simultaneously or sequentially administering an additional active agent for treatment of retinitis pigmentosa.

In some embodiments, the amount sufficient to inhibit neurodegeneration is an amount sufficient to reduce an amount or rate of apoptosis of a population of neurons contacted with the composition. In some embodiments, the neuron is subject to elevated hydrostatic pressure and the method comprises contacting the neuron with the composition comprising cannabinol, or a derivative thereof, in an amount sufficient to reduce pressure-induced neurodegeneration.

In some embodiments, the amount sufficient to inhibit neurodegeneration is an amount that results in a concentration of from about 0.15 μM to less than about 15 μM of cannabinol (or a derivative thereof, such as cannabinolic acid, or a prodrug thereof) in contact with the target neuron, target neuronal population, or in the ocular tissues of the eye. In some embodiments, the amount sufficient to inhibit neurodegeneration is an amount that results in a concentration of greater than about 0.5 μM and less than 15 μM of cannabinol in contact with the target neuron, target neuronal population, or in the ocular tissues of the eye.

In some embodiments, the amount sufficient to inhibit neurodegeneration is an amount that results in a concentration of from about 0.5 μM to less than 15 μM of cannabinol (or a derivative thereof, such as cannabinolic acid, or a prodrug thereof), preferably from greater than about 0.5 μM to less than 12 μM of cannabinol (or a derivative thereof, such as cannabinolic acid, or a prodrug thereof) in contact with the target neuron, target neuronal population, or in the ocular tissues of the eye. In some embodiments, the amount sufficient to inhibit neurodegeneration is an amount that results in a concentration of from about 1.5 μM to 10 μM of cannabinol (or a derivative thereof, such as cannabinolic acid, or a prodrug thereof) in contact with the target neuron, target neuronal population, or in the ocular tissues of the eye.

In some embodiments, the cannabinol is provided in an extended release formulation. In some embodiments, the formulation comprises: a) a delivery carrier comprising a cellulosic polymer and an anionic polysaccharide; and b) nanoparticles comprising an amphiphilic non-ionizable block copolymer and cannabinol, wherein the formulation has a gel point of about 30° C. to about 37° C.

In some embodiments, the contacting comprises systemically administering the composition comprising cannabinol or a prodrug thereof, or a derivative thereof such as cannabinolic acid or a prodrug thereof. In some embodiments, the systemic administration comprises intravenous injection. In some embodiments, the systemic administration comprises oral administration. In some embodiments, the systemic administration comprises transdermal administration.

In some embodiments, the contacting comprises local administration of the composition comprising cannabinol or a prodrug thereof, or a derivative thereof such as cannabinolic acid or a prodrug thereof. In some cases, the contacting comprises administering the composition comprising cannabinol or a prodrug thereof, or a derivative thereof such as cannabinolic acid or a prodrug thereof, directly to the eye. For example, the contacting can comprise administering the composition comprising cannabinol or a prodrug thereof, or a derivative thereof such as cannabinolic acid or a prodrug thereof, onto the eye (e.g., as an eyedrop such as in the form of a microemulsion eyedrop, or an eyegel). As another example, the contacting can comprise administering the composition comprising cannabinol or a prodrug thereof, or a derivative thereof such as cannabinolic acid or a prodrug thereof, directly into the eye (e.g., via intravitreal injection or pump).

In another aspect, described herein is a composition for treatment of neurodegeneration in a subject comprising cannabinol, or a derivative thereof. In some embodiments, the composition is a pharmaceutical formulation suitable to achieve a neuroprotective dose of cannabinol, or a derivative thereof. In some embodiments, the composition is formulated for administration to the eye. In some embodiments, the composition is formulated to achieve a concentration of from about 0.15 μM to less than about 15 μM cannabinol, or derivative thereof, in the ocular tissues of the eye and/or in contact with retinal ganglions.

In another aspect, described herein is a use of a composition comprising cannabinol, or a derivative thereof, for treatment of neurodegeneration in a subject, such as hydrostatic pressure induced neurodegeneration, preferably according to one or more of the foregoing aspects, embodiments, cases, or examples, with a composition described herein, or according to a method described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

FIG. 1 illustrates in vitro neuroprotection of differentiated 661W retinal ganglion precursor-like cells cultured under atmospheric pressure by contacting the cells with CBD, CBDA, CBC, CBG, CBGA, CBN, CBND, Δ⁹-THC at 0.5 μM, 1.5 μM and 5 μM respectively for each cannabinoid. Vehicle Control (VC) contained 0.15% ethanol. Data presented as Cell Death (%) vs Vehicle Control (taken as 0%). Treatment conditions: 72 hours at concentrations of 0.5, 1.5 and 5 μM for different Cannabinoids, or vehicle control (VC).

FIG. 2 illustrates an absence of significant neuroprotection of differentiated 661W cells when cultured in a pressurized chamber with elevated hydrostatic pressure of approximately 20 to 40 mm Hg by contacting cells with CBD, CBDA, Δ⁹-THC, CBGA, or CBND at 0.5 μM, 1.5 μM and 5 μM treatment concentrations respectively for each cannabinoid, or vehicle control (VC), for 72 hours. Vehicle Control (VC) contained 0.15% ethanol. Data presented as Cell Death (%) vs Normal Pressure Vehicle Control (taken as 0% cell death).

FIG. 3 illustrates statistically significant neuroprotection of differentiated 661W cells when cultured in a pressurized chamber with elevated hydrostatic pressure of approximately 20 to 40 mm Hg by contacting cells for 72 hours with vehicle control (VC) containing 0.15% ethanol or CBN at 0.015 μM, 0.05 μM, 0.15 μM, 0.5 μM, 1.5 μM, 5 μM, 10 μM and 15 μM as indicated. Data presented as Cell Death (%) vs a Normal Pressure Vehicle Control (taken as 0% cell death). Statistically significant difference compared to Vehicle Control (VC) by One-Way ANOVA, (Dunnett's multiple comparison's test).

FIG. 4 illustrates a comparison of the significant neuroprotective effect of CBN on differentiated 661W cells when cultured for 72 hours under a hydrostatic pressure of approximately 20 to 40 mm Hg as compared to Δ⁹-THC. CBN at 0.5 μM, 1.5 μM, 5 μM, 10 μM and 15 μM treatment concentrations and Δ⁹-THC at 0.5 μM, 1.5 μM and 5 μM respectively. Vehicle Control (VC) contained 0.15% ethanol. Data presented as Cell Death (%) vs Normal Pressure Vehicle Control (taken as 0% cell death). Statistically significant difference compared to Vehicle Control (VC) by One-Way ANOVA (Dunnett's multiple comparison's test).

FIG. 5 illustrates a comparison of the significant neuroprotective effect of CBN and CBD on differentiated 661W cells when cultured for 72 hours under a hydrostatic pressure of approximately 20 to 40 mm Hg. CBN at 0.5 μM, 1.5 μM, 5 μM, 10 μM and 15 μM and CBD at 0.5 μM, 1.5 μM and 5 μM respectively, or vehicle control (VC). Vehicle Control (VC) contained 0.15% ethanol. Data presented as Cell Death (%) vs Normal Pressure Vehicle Control (taken as 0% cell death). Statistically significant difference compared to Vehicle Control (VC) by One-Way ANOVA (Dunnett's multiple comparison's test).

FIG. 6 illustrates statistically significant neuroprotection of differentiated 661W cells when cultured inside pressurized chamber with an elevated hydrostatic pressure of approximately 10 to 25 mmHg by contacting cells with a cannabinol-derivative CBNA having the following formula:

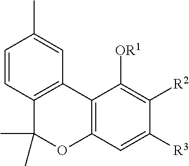

wherein: R¹ is H, R² is COOH, and R³ is n-C₅H₁₁. Vehicle Control (VC) contained 0.15% ethanol. Data presented as Cell Death (%) vs Vehicle Control (taken as 0%). Treatment conditions: 72 hours at concentrations 0.015, 0.05, 0.15, 0.5, 1.5, 5, 10 and 15 μM, or vehicle control. Statistically significant difference compared to Vehicle Control (VC) by One-Way ANOVA (Dunnett's multiple comparison's test).

FIG. 7 illustrates Cannabinol's protective effect of 661W cells from apoptosis when cultured under elevated pressure. Treatment of 661W cells with Vehicle Control (VC) containing no cannabinoid under elevated pressure resulted in approximately 35% induction of apoptosis. Treatment of 661W cells with Cannabinol was able to protect neuronal cells from apoptosis at concentrations greater than 0.015 μM and less than 5 μM, with a statistically significant protective effect in the range between 0.05 and 1.5 μM (p<0.05 and p<0.01). Vehicle Control (VC) contained 0.15% ethanol. Data presented as Apoptosis (%) vs Normal Pressure Vehicle Control (taken as 0%). Statistically significant difference compared to Vehicle Control (VC) by One-Way ANOVA (Dunnett's multiple comparison's test).

FIG. 8 illustrates Cannabinol's neuroprotective effect on RGCs by measuring pattern electroretinogram (pERG) amplitudes in the rat episcleral vein laser photocoagulation model of glaucoma. The functional response of RGCs measured by reduction of pERG amplitudes decreased in all treatment groups after the TOP elevation induced by laser treatments. Statistically significant impairment of RGC function was observed in the Vehicle-treated group at day 21 and in CBN (high dose) group at days 14 and 21 (two-way ANOVA followed by Tukey's multiple comparison test, *p<0.05). The pERG amplitudes in the CBN (low dose) group at 5 μM final concentration inside the eye and Brimonidine (ALPHAGAN) group did not differ significantly from the baseline on both follow-up days 14 and 21 indicating that CBN at low doses confers neuroprotective effect on RGCs similar to ALPHAGAN.

### DETAILED DESCRIPTION OF THE INVENTION

Described herein are methods and compositions for protecting neurons from one or more cytotoxic stimuli. In some embodiments, the method includes contacting the neurons with the neuroprotective composition, such as by administering the composition to a subject in need thereof. The methods and compositions described herein find particular use, but are not limited to, protection of retinal neurons. In some cases, the methods and compositions described herein can be used for neuroprotection of retinal neurons in a subject, such as a subject suffering from glaucoma or increased intraocular pressure as compared to normal intraocular pressure, e.g., in a normal subject. In certain embodiments, the neuroprotective composition is a cannabinoid, such as cannabinol. In some cases, the method includes contacting retinal neurons with the neuroprotective agent (e.g., cannabinol), such as by administering the neuroprotective agent to a subject in need thereof. In some cases, the neuroprotective composition is or contains cannabinol or a solvate thereof.

In some cases, the neuroprotective composition is or contains a cannabinol derivative, such as a derivative described in U.S. 2003/0158191, a salt thereof, or a solvate thereof. In an embodiment, the neuroprotective composition is or contains a cannabinol derivative compound as claimed in U.S. Pat. No. 7,105,685. In an embodiment, the neuroprotective composition is or contains a cannabinol derivative compound selected from the group consisting of the cannabinol-type (CBN-type) cannabinoids described in ElSohly & Slade, Life Sciences, 78 (2005), p. 539-48. For example, the neuroprotective composition can be or contain cannabinol or a derivative having the formula of Formula I:

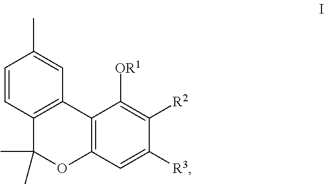

wherein: R¹ is H, R² is COOH, and R³ is n-C₅H₁₁;
R¹ is H, R² is H, and R³ is n-C₅H₁₁;
R¹ is CH₃, R² is H, and R³ is n-C₅H₁₁;
R¹ is H, R² is H, and R³ is n-C₄H₉;
R¹ is H, R² is H, and R³ is n-C₃H₇;
R¹ is H, R² is H, and R³ is C₂H₅; or
R¹ is H, R² is H, and R³ is CH₃.

In some embodiments, the neuroprotective composition can be or contain a derivative having the formula of Formula I, wherein R¹ is H, R² is COOH, and R³ is n-C₅H₁₁.

In some embodiments, the neuroprotective composition can contain a prodrug of any one of the cannabinols or derivatives thereof described herein. For example, the neuroprotective composition can contain a prodrug of cannabinol, or a derivative thereof. As another example, the neuroprotective composition can contain a prodrug of a derivative having the formula of Formula I. As another example, the neuroprotective composition can contain a prodrug of a derivative having the formula of Formula I, wherein R¹ is H, R² is COOH, and R³ is n-C₅H₁₁; R¹ is H, R² is H, and R³ is n-C₅H₁₁; R¹ is CH₃, R² is H, and R³ is n-C₅H₁₁; R¹ is H, R² is H, and R³ is n-C₄H₉; R¹ is H, R² is H, and R³ is n-C₃H₇; R¹ is H, R² is H, and R³ is C₂H₅; or R¹ is H, R² is H, and R³ is CH₃. In some cases, the neuroprotective composition contains a prodrug of a derivative having the formula of Formula I, wherein R¹ is H, R² is COOH, and R³ is n-C₅H₁₁.

In some cases, the prodrug is an ester of cannabinol or a derivative thereof. In some cases, the prodrug is an ester of a derivative having a formula of Formula I, such as one of the derivatives described herein. In some cases, the prodrug is a D-(−)-glyceric acid ester of cannabinol or a derivative thereof. In some cases, the prodrug is a D-(−)-glyceric acid ester of cannabinolic acid or a derivative thereof. In some cases, the prodrug is a D-(−)-glyceric acid ester of a derivative having a formula of Formula I, such as one of the derivatives described herein. Additional prodrug strategies for the neuroprotective compounds described herein can be found in U.S. Pat. Publ. Nos. 2016/0228490; 2011/0052694; 2015/0197484; 2008/0076789; 2009/0143462; 2012/0289484; 2009/0036523; 2009/0156814; and 2008/0008745; and Adelli et al., Investigative Opthalmology & Visual Science, April 2017, Vol. 58, No. 4, p. 2168; and Upadhye et al., AAPS PharmSciTech, Vol. 11, No. 2, June 2010, p. 509, the contents of which are hereby incorporated in the entirety for all purposes and in particular for the cannabinoid prodrug compositions and formulations, and methods of making, using and/or administering such prodrug compositions described therein.

The neuroprotective composition can contain additional active agents. In some embodiments, the neuroprotective composition can contain cannabinol, or a derivative thereof, and an additional cannabinoid or a terpenoid. In some embodiments, the neuroprotective composition can contain an additional active pharmaceutical agent for treatment of glaucoma or an additional active pharmaceutical agent for treatment of increased intraocular pressure.

Currently, different classes of therapeutic agents are used for the reduction of intraocular pressure and/or treatment of glaucoma, including but not limited to: a prostaglandin analog, a β-adrenergic antagonist, an α-adrenergic agonist, a carbonic anhydrase inhibitor, and/or a cholinergic agonist.

Prostaglandin analogs include but are not limited to latanoprost, travoprost, tafluprost, unoprostone, or bimatoprost. Prostaglandin analogs are therapeutic agents that increase the uveoscleral outflow of aqueous humor. These drugs are typically administered once a day, at night, and this restricts the action of pressure reduction to the night period only. They have many side effects, both local and systemic, including conjunctival hyperemia, thickening of the eyelashes, iris coloring, uveitis, macular edema and headache.

β-adrenergic antagonists include but are not limited to timolol, levobunolol, carteolol, metipranolol, or betaxolol. The mechanism by which these drugs act consists in the reduction of the production of aqueous humor. They are typically administered once a day, in the morning, and have serious systemic side effects due to the antagonistic action on β-adrenergic receptors. This restricts the possibilities for use in patients with asthma, chronic obstructive pulmonary disease and bradycardia.

α-adrenergic agonists include but are not limited to brimonidine, or apraclonidine. These drugs lead to initial reduction in the production of aqueous humor and increase in the outflow of the latter. In this case too, numerous side effects, both local and systemic, occur, such as irritation and eye dryness, allergic reactions, effects on the central nervous system, respiratory arrest, postural hypotension, brain or coronary failure, liver and kidney damage. They must typically be administered 3 times a day and this reduces patient compliance.

Carbonic anhydrase inhibitors include but are not limited to dorzolamide, brinzolamide, or acetazolamide. These drugs reduce the production of aqueous humor. Side effects include eye irritation, eye burning, paraesthesia, nausea, diarrhea, loss of appetite.

Cholinergic agonists include but are not limited to pilocarpine, or carbachol. These drugs increase the outflow of aqueous humor. They are typically administered more than 4 times a day, with considerable reduction in patient compliance and consequent reduced effectiveness due to poor adherence to the therapeutic scheme. Side effects also occur in this case, including eye irritation, myopia, ciliary spasm, smaller pupils, blurred or dim vision, and nearsightedness with consequent headache and loss of vision.

In some embodiments, the neuroprotective compositions described herein, e.g., containing cannabinol or a derivative thereof, allow a lower dose, or less frequent dosing, of one or more therapeutic agents for treatment of glaucoma.

### Definitions

As used herein, “a subject in need thereof,” and the like, refers to a mammal, preferably a human.

As used herein, “cannabinol” or “CBN” refers to 6,6,9-trimethyl-3-pentylbenzo[c]chromen-1-ol.

“Salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

As used herein, the term “solvate” means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate.” Examples of hydrate include, but are not limited to, hemihydrate, monohydrate, dihydrate, trihydrate, hexahydrate, and other water-containing species. It should be understood by one of ordinary skill in the art that the pharmaceutically acceptable salt, and/or prodrug of a compound may also exist in a solvate form. The solvate is typically formed via hydration which is either part of the preparation of a compound or through natural absorption of moisture by an anhydrous compound of the present invention. In general, all physical forms are intended to be within the scope of the present invention.

Thus, when a therapeutically active agent made in a method according to the present invention or included in a composition according to the present invention, such as, but not limited to, a cannabinol derivative, possesses a sufficiently acidic, a sufficiently basic, or both a sufficiently acidic and a sufficiently basic functional group, this group or groups can accordingly react with any of a number of inorganic or organic bases, and inorganic and organic acids, to form a pharmaceutically acceptable salt. Exemplary pharmaceutically acceptable salts include those salts prepared by reaction of the pharmacologically active compound with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, (3-hydroxybutyrates, glycolates, tartrates, methane-sulfonates, propanesulfonates, naphthalene-1-sulfonates, naphthalene-2-sulfonates, and mandelates. If the pharmacologically active compound has one or more basic functional groups, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, or with a pyranosidyl acid, such as glucuronic acid or galacturonic acid, or with an alpha-hydroxy acid, such as citric acid, tartaric acid, or with an amino acid, such as aspartic acid, glutamic acid, or with an aromatic acid, such as benzoic acid, cinnamic acid, or with a sulfonic acid, such as p-toluenesulfonic acid or ethanesulfonic acid, or the like. If the pharmacologically active compound has one or more acidic functional groups, the desired pharmaceutically acceptable salt may be prepared by any suitable method available in the art, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

“Composition” as used herein is intended to encompass a product comprising the specified ingredients in the specified amounts, as well as any product that results from combination of the specified ingredients in the specified amounts. By “pharmaceutically acceptable” it is meant the carrier, diluent or excipient must be compatible with the other ingredients of the formulation and not deleterious to the recipient thereof.

“Pharmaceutically acceptable excipient” refers to a substance that aids the administration of an active agent to the subject and/or absorption by a subject. Pharmaceutical excipients useful in the present invention include, but are not limited to binders, fillers, disintegrants, lubricants, coatings, sweeteners, flavors and colors. One of skill in the art will recognize that other pharmaceutical excipients are useful in the present invention.

In some cases, protecting groups can be included in compounds used in methods according to the present invention or in compositions according to the present invention. The use of such a protecting group is to prevent subsequent hydrolysis or other reactions that can occur in vivo and can degrade the compound. Groups that can be protected include alcohols, amines, carbonyls, carboxylic acids, phosphates, and terminal alkynes. Protecting groups useful for protecting alcohols include, but are not limited to, acetyl, benzoyl, benzyl, β-methoxyethoxyethyl ether, dimethoxytrityl, methoxymethyl ether, methoxytrityl, ρ-methoxybenzyl ether, methylthiomethyl ether, pivaloyl, tetrahydropyranyl, tetrahydrofuran, trityl, silyl ether, methyl ether, and ethoxyethyl ether. Protecting groups useful for protecting amines include carbobenzyloxy, p-methoxybenzylcarbonyl, t-butyloxycarbonyl, 9-fluorenylmethyloxycarbonyl, acetyl, benzoyl, benzyl, carbamate, p-methoxybenzyl, 3,4-dimethoxybenzyl, p-methoxyphenyl, tosyl, trichloroethyl chloroformate, and sulfonamide. Protecting groups useful for protecting carbonyls include acetals, ketals, acylals, and dithianes. Protecting groups useful for protecting carboxylic acids include methyl esters, benzyl esters, t-butyl esters, esters of 2,6-disubstituted phenols, silyl esters, orthoesters, and oxazoline. Protecting groups useful for protecting phosphate groups include 2-cyanoethyl and methyl. Protecting groups useful for protecting terminal alkynes include propargyl alcohols and silyl groups. Other protecting groups are known in the art.

As used herein, the term “prodrug” refers to a derivative that is a precursor compound that, following administration, releases the biologically active compound in vivo via some chemical or physiological process (e.g., a prodrug on reaching physiological pH or through enzyme action is converted to the biologically active compound). A prodrug itself may either lack or possess the desired biological activity. Thus, the term “prodrug” refers to a precursor of a biologically active compound that is pharmaceutically acceptable. In certain cases, a prodrug has improved physical and/or delivery properties over a parent compound from which the prodrug has been derived. The prodrug often offers advantages of solubility, tissue compatibility, or delayed release in a mammalian organism (H. Bundgard, Design of Prodrugs (Elsevier, Amsterdam, 1988), pp. 7-9, 21-24). A discussion of prodrugs is provided in T. Higuchi et al., “Pro-Drugs as Novel Delivery Systems,” ACS Symposium Series, Vol. 14 and in E.B. Roche, ed., Bioreversible Carriers in Drug Design (American Pharmaceutical Association & Pergamon Press, 1987). Exemplary advantages of a prodrug can include, but are not limited to, its physical properties, such as enhanced drug stability for long-term storage.

The term “prodrug” is also meant to include any covalently bonded carriers which release the active compound in vivo when the prodrug is administered to a subject. Prodrugs of a therapeutically active compound, as described herein, can be prepared by modifying one or more functional groups present in the therapeutically active compound, including cannabinoids, such as cannabinol, or a cannabinol derivative, and other therapeutically active compounds used in methods according to the present invention or included in compositions according to the present invention, in such a way that the modifications are cleaved, either in routine manipulation or in vivo, to yield the parent therapeutically active compound. Prodrugs include compounds wherein a hydroxy, amino, or mercapto group is covalently bonded to any group that, when the prodrug of the active compound is administered to a subject, cleaves to form a free hydroxy, free amino, or free mercapto group, respectively. Examples of prodrugs include, but are not limited to, formate or benzoate derivatives of an alcohol or acetamide, formamide or benzamide derivatives of a therapeutically active agent possessing an amine functional group available for reaction, and the like. In some cases, the prodrug is a protecting group modified derivative of the neuroprotective compound, such as a protecting group modified cannabinol or a protecting group modified derivative of cannabinol.

For example, if a therapeutically active agent or a pharmaceutically acceptable form of a therapeutically active agent contains a carboxylic acid functional group, a prodrug can comprise an ester formed by the replacement of the hydrogen atom of the carboxylic acid group with a group such as C₁₋₈ alkyl, C₂₋₁₂ alkanoyloxymethyl, 1-(alkanoyloxy)ethyl having from 4 to 9 carbon atoms, 1-methyl-1-(alkanoyloxy)ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N(C₁-C₂)alkylamino(C₂-C₃)alkyl (such as (3-dimethylaminoethyl), carbamoyl-(C₁-C₂)alkyl, N,N-di (C₁-C₂)alkylcarbamoyl-(C₁-C₂)alkyl and piperidino-, pyrrolidino-, or morpholino(C₂-C₃)alkyl.

In some cases, the therapeutically active agent or a pharmaceutically acceptable form of a therapeutically active agent is a cannabinoid, such as a cannabinoid of Formula I, that contains an H at R², and the prodrug comprises a 3,6,9,12-tetraoxatridecanoyl ester; an N,N-dimethylglycyl ester; a 3,6,9,12-tetraoxatridecyl carbonate; an N-formulglycyl ester; an N-formylsarcosyl ester; a 3,6,9,12-tetraoxatridecyl oxalate; a hemisuccinate; a 4-aminobutyl carbamate; a prolyl ester; a 3-dimethylamino propionate; a glycolate; a (D)-Ribonate; a phosphate ammonium salt; an (R)-2,3-dihydroxypropyl carbonate; a 3-hydroxy-2-(hydroxymethyl)-2-methylpropanoate; a glycinate; a f3-alaninate; an (S)-2,3-dihydroxypropanoate; an (S)-2,3-dihydroxypropyl carbonate; or an (R)-2,3-dihydroxypropyl carbonate at R¹.

In some cases, the therapeutically active agent or a pharmaceutically acceptable form of a therapeutically active agent is a cannabinoid, such as a cannabinoid of Formula I, that contains a carboxylic acid functional group at R², and the prodrug comprises a 3,6,9,12-tetraoxatridecanoyl ester; an N,N-dimethylglycyl ester; a 3,6,9,12-tetraoxatridecyl carbonate; an N-formulglycyl ester; an N-formylsarcosyl ester; a 3,6,9,12-tetraoxatridecyl oxalate; a hemisuccinate; a 4-aminobutyl carbamate; a prolyl ester; a 3-dimethylamino propionate; a glycolate; a (D)-Ribonate; a phosphate ammonium salt; an (R)-2,3-dihydroxypropyl carbonate; a 3-hydroxy-2-(hydroxymethyl)-2-methylpropanoate; a glycinate; a f3-alaninate; an (S)-2,3-dihydroxypropanoate; an (S)-2,3-dihydroxypropyl carbonate; or an (R)-2,3-dihydroxypropyl carbonate derivative at R¹.

In some cases, the prodrug is a prodrug of CBNA (COOH at R² of Formula I, n-C₅H₁₁ at R³) comprising an ester, a carbonate, a carbamate, or a phosphate, such as one of the foregoing esters, carbonates, carbamates, or phosphates, at R¹.

Similarly, if a disclosed compound or a pharmaceutically acceptable form of the compound contains an alcohol functional group, a prodrug can be formed by the replacement of the hydrogen atom of the alcohol group with a group such as (C₁-C₆)alkanoyloxymethyl, 1 -((C₁-C₆))alkanoyloxy)ethyl, 1-methyl-1-((C₁-C₆)alkanoyloxy)ethyl (C₁-C₆)alkoxycarbonyloxymethyl, N(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, P(O)(O(C₁-C₆)alkyl)₂ or glycosyl (the radical resulting from the removal of a hydroxyl group of the hemiacetal form of a carbohydrate).

If a disclosed compound or a pharmaceutically acceptable form of the compound incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, C(OH)C(O)OY¹ wherein Y¹ is H, (C₁-C₆)alkyl or benzyl, C(OY²)Y³ wherein Y² is (C₁-C₄) alkyl and Y³ is (C₁-C₆)alkyl, carboxy(C₁-C₆)alkyl, amino(C₁-C₄)alkyl or mono-N or di-N,N(C i-C₆)alkylaminoalkyl,C(Y⁴)Y⁵ wherein Y⁴ is H or methyl and Y⁵ is mono-N or di-N,N(C i-C₆)alkylamino, morpholino, piperidin-1-yl or pyrrolidin-1-yl.

The use of prodrug systems is described in T. Järvinen et al., “Design and Pharmaceutical Applications of Prodrugs” in Drug Discovery Handbook (S. C. Gad, ed., Wiley-Interscience, Hoboken, N.J., 2005), ch. 17, pp. 733-796. Other alternatives for prodrug construction and use are known in the art. When a method or pharmaceutical composition according to the present invention, uses or includes a prodrug of cannabinol, or other therapeutically active agent, prodrugs and active metabolites of a compound may be identified using routine techniques known in the art. See, e.g., Bertolini et al., J. Med. Chem., 40, 2011-2016 (1997); Shan et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992).

As used herein, the terms “therapeutically effective quantity,” “therapeutically effective dose,” or “therapeutically effective amount” refer to a dose of one or more compositions described herein that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

### Cannabinoids

Cannabinoids are a group of chemicals known to activate cannabinoid receptors in cells throughout the human body, including the skin. Phytocannabinoids are the cannabinoids derived from cannabis plants. They can be isolated from plants or produced synthetically. Endocannabinoids are endogenous cannabinoids produced naturally by cells in the human body. Canonical phytocannabinoids are ABC tricyclic terpenoid compounds bearing a benzopyran moiety.

Cannabinoids exert their effects by interacting with cannabinoid receptors present on the surface of the cells. To date, two types of cannabinoid receptor have been identified, the CB1 receptor and the CB2 receptor. These two receptors share about 48% amino acid sequence identity and are distributed in different tissues and have distinct cell signaling mechanisms. They also differ in their sensitivity to agonists and antagonists.

In some cases, the cannabinoids or precursors thereof, can be purified, derivatized (e.g., to form a prodrug, solvate, or salt, or to form a target cannabinoid from the precursor), and/or formulated in a pharmaceutical composition.

Cannabinoids include but are not limited to phytocannabinoids. In some cases the cannabinoids include but are not limited to, cannabinol, cannabidiols, Δ⁹-tetrahydrocannabinol (Δ⁹-THC), the synthetic cannabinoid HU-210 (6aR,10aR)-9-(hydroxymethyl)-6,6-dimethyl-3-(2-methyloctan-2-yl)-6H,6aH,7H,10H,10aH-benzo[c]isochromen-1-ol), HU-308 ([(1R,2R,5R)-2-[2,6-dimethoxy-4-(2-methyloctan-2-yl)phenyl]-7,7-dimethyl-4-bicyclo[3.1.1]hept-3-enyl]methanol), HU-433 an enantiomer of HU-308, cannabidivarin (CBDV), cannabichromene (CBC), cannabichromevarin (CBCV), cannabigerol (CBG), cannabigerovarin (CBGV), cannabielsoin (CBE),cannabicyclol (CBL),cannabivarin (CBV), and cannabitriol (CBT). Still other cannabinoids include, including tetrahydrocannibivarin (THCV) and cannabigerol monomethyl ether (CBGM). Additional cannabinoids include cannabichromenic acid (CBCA), Δ⁹-tetrahydrocannabinolic acid (THCA); and cannabidiolic acid (CBDA); these additional cannabinoids are characterized by the presence of a carboxylic acid group in their structure.

Still other cannabinoids include nabilone, rimonabant, JWH-018 (naphthalen-1-yl-(1-pentylindol-3-yl)methanone), JWH-073 naphthalen-1-yl-(1-butylindol-3-yl)methanone, CP-55940 (2-[(1R,2R,5R)-5-hydroxy-2-(3-hydroxypropyl) cyclohexyl]-5-(2-methyloctan-2-yl)phenol), dimethylheptylpyran, HU-331 (3-hydroxy-2-[(1R)-6-isopropenyl-3-methyl-cyclohex-2-en-1-yl]-5-pentyl-1,4-benzoquinone), SR144528 (5-(4-chloro-3-methylphenyl)-1-[(4-methylphenyl)methyl]-N-R¹S,2S,4R)-1,3,3-trimethylbicyclo[2.2.1]heptan-2-yl1-1H-pyrazole-3-carboxamide), WIN 55,212-2 ((11R)-2-methyl-11-[(morpholin-4-yl)methyl]-3-(naphthalene-1-carbonyl)-9-oxa-1-azatricyclo[6.3.1.0⁴,¹²1 dodeca-2,4(12),5,7-tetraene), JWH-133 ((6aR,10aR)-3-(1,1-dimethylbutyl)-6a,7,10,10a-tetrahydro-6,6,9-trimethyl-6H-dibenzo[b,d]pyran),levonatradol, and AM-2201 (1-[(5-fluoropentyl)-1H-indol-3-yl]-(naphthalen-1-yl)methanone). Other cannabinoids include Δ⁸-tetrahydrocannabinol (Δ⁸-THC), 11-hydroxy-Δ⁹-tetrahydrocannabinol, Δ¹¹-tetrahydrocannabinol, and 11-hydroxy-tetracannabinol.

In another alternative, analogs or derivatives of these cannabinoids can be obtained by providing a precursor cannabinoid and further derivatization, e.g. , by synthetic means. Synthetic cannabinoids include, but are not limited to, those described in U.S. Pat. No. 9,394,267 to Attala et al.; U.S. Pat. No. 9,376,367 to Herkenroth et al.; U.S. Pat. No. 9,284,303 to Gijsen et al.; U.S. Pat. No. 9,173,867 to Travis; U.S. Pat. No. 9,133,128 to Fulp et al.; U.S. Pat. No. 8,778,950 to Jones et al.; U.S. Pat. No. 7,700,634 to Adam-Worrall et al.; U.S. Pat. No. 7,504,522 to Davidson et al.; U.S. Pat. No. 7,294,645 to Barth et al.; U.S. Pat. No. 7,109,216 to Kruse et al.; U.S. Pat. No. 6,825,209 to Thomas et al.; and U.S. Pat. No. 6,284,788 to Mittendorf et al.

Neuroprotective cannabinoids according to the present invention can be at least partially selective for binding to either the CB2 cannabinoid receptor or the CB1 cannabinoid receptor. In some embodiments, the neuroprotective cannabinoids bind both the CB1 and CB2 cannabinoid receptors. In some cases, neuroprotective cannabinoids according to the present invention are selective for the CB1 cannabinoid receptor and act as partial agonists. In some other cases, neuroprotective cannabinoids according to the present invention are selective for the CB2 cannabinoid receptor and act as partial agonists. In some cases, the neuroprotective cannabinoids bind to both CB1 and CB2 receptors acting as partial agonists for both receptors but with higher affinity to CB2 receptor at a similar potency to that of Δ⁹-THC. In some cases, the cannabinoids, or one of the cannabinoids in a mixture of neuroprotective cannabinoids is an inverse agonist of CB2 receptor. As an inverse agonist, the neuroprotective cannabinoids can bind to the CB2 receptor but can induce a pharmacological response opposite to that of agonist. In some cases, cannabinoids in the neuroprotective compositions and methods according to the present invention are partially selective for the CB2 cannabinoid receptor. In some cases, the neuroprotective cannabinoid or mixture of cannabinoids exhibit, e.g., at least, a 3-fold lower Ki against CB2 receptor as compared to CB1 receptor in an in vitro competition assay with an overall higher binding affinity to CB2.

An exemplary cannabinoid is cannabinol or cannabinolic acid.

Exemplary prodrugs useful in the present invention include but are not limited to the following prodrugs of cannabinol (left) and cannabinolic acid (right):

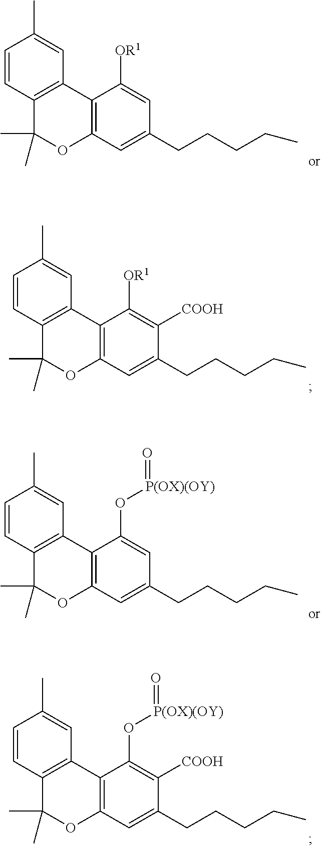

wherein X and Y, can be the same or different, and are selected from the group consisting of: hydrogen, alkali metals (e.g., sodium and potassium), alkaline earth metals (e.g., calcium and magnesium); and cations of pharmaceutically acceptable organic amines (e.g., quaternated or protonated amines, including alkyl amines, hydroxyalkylamines, monoamines, diamines, and naturally occurring amines). Examples of such pharmaceutically acceptable organic bases include choline, betaine, caffeine, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, hydrabamine, isopropylamine, methylglucamine, morpholine, piperidine, polyamine resins, procaine, purines, theobromine, triethylamine, trimethylamine, tripropylamine, tromethamine, tetramethylammonium hydroxide, benzyltrimethylammonium hydroxide, tris(hydroxymethyl)aminomethane (TRIS), N-(2-hydroxyethyl)pyrrolidine, piperazine, glucosamine, arginine, lysine and histidine. In a further embodiment, X and Y are different substituent groups. In another embodiment, X and Y are the same substituent group. In a further embodiment, X and Y can both be part of the same functional group, such as piperazine. In a further embodiment, the phosphate is selected from a group consisting of a diphosphate and triphosphate. In another embodiment, the compound is the salt form of the di or tri phosphate;

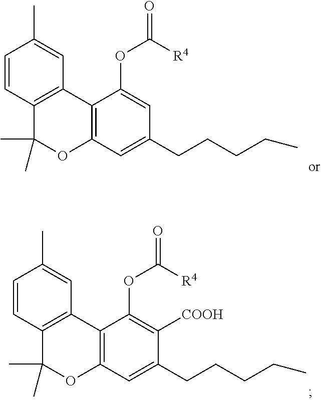

wherein R⁴ is a straight or branched chain substituted or unsubstituted alkyl or alkoxyalkyl, akylamine, hydroxyalkyl, or hydroxyalkylamine, preferably wherein R⁴ comprises from 1 to 12 carbons and optionally no more than 4 substitutions, more preferably wherein R⁴ comprises from 1 to 6 carbons and optionally no more than 2 substitutions;

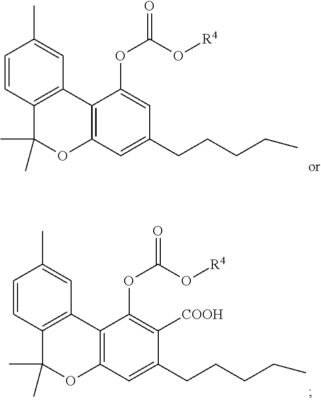

wherein R⁴ is a straight or branched chain substituted or unsubstituted alkyl or alkoxyalkyl, akylamine, hydroxyalkyl, or hydroxyalkylamine, preferably wherein R⁴ comprises from 1 to 12 carbons and optionally no more than 4 substitutions, more preferably wherein R⁴ comprises from 1 to 6 carbons and optionally no more than 2 substitutions;

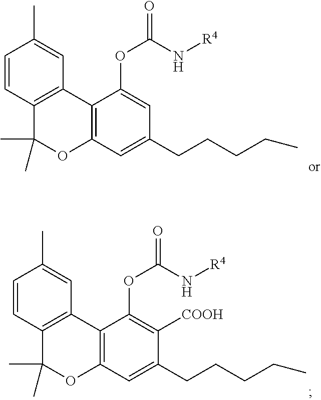

wherein R⁴ is a straight or branched chain substituted or unsubstituted alkyl or alkoxyalkyl, akylamine, hydroxyalkyl, or hydroxyalkylamine, preferably wherein R⁴ comprises from 1 to 12 carbons and optionally no more than 4 substitutions, more preferably wherein R⁴ comprises from 1 to 6 carbons and optionally no more than 2 substitutions;

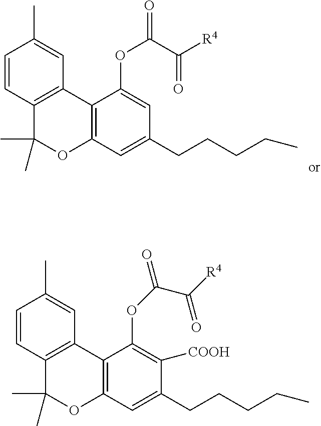

wherein R⁴ is a straight or branched chain substituted or unsubstituted alkyl or alkoxyalkyl, akylamine, hydroxyalkyl, or hydroxyalkylamine, preferably wherein R⁴ comprises from 1 to 12 carbons and optionally no more than 4 substitutions, more preferably wherein R⁴ comprises from 1 to 6 carbons and optionally no more than 2 substitutions.

In some embodiments, prodrugs useful in the present invention include but are not limited to the following prodrugs of cannabinol (left) and cannabinolic acid (right):

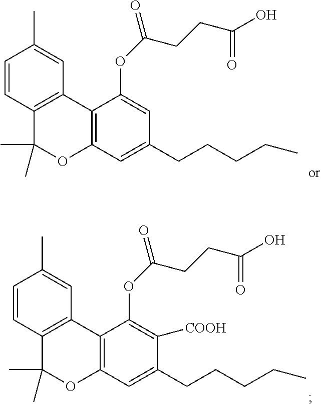

In some embodiments, the foregoing prodrugs may be advantageously formulated with a cyclodextrin, such as random methylated beta-cyclodextrin, 2-hydroxypropyl beta-cyclodextrin, or sulfobutyl ether beta-cyclodextrin.

In some embodiments, prodrugs useful in the present invention include but are not limited to the following prodrugs of cannabinol (left) and cannabinolic acid (right):

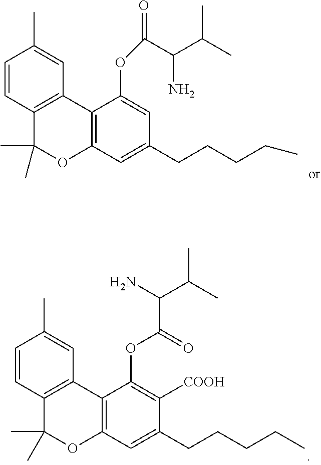

In some embodiments, prodrugs useful in the present invention include but are not limited to the following prodrugs of cannabinol:

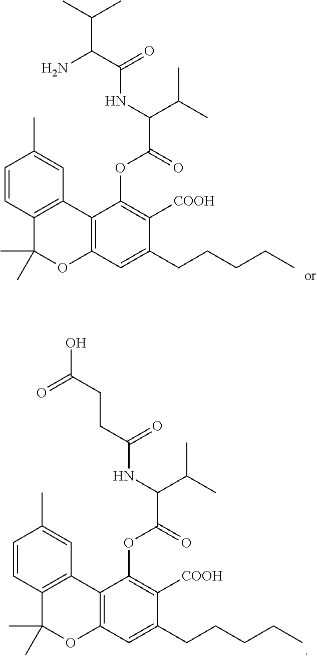

In some embodiments, prodrugs useful in the present invention include but are not limited to the following prodrugs of cannabinolic acid:

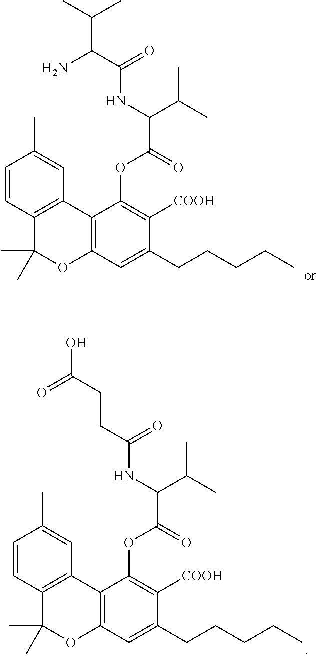

### Pharmaceutical Compositions

The compositions described herein are typically formulated for administration. Accordingly, described herein is a composition comprising cannabinol formulated for administration with one or more and a pharmaceutically acceptable carrier(s), diluent(s), or excipient(s).

The pharmaceutical compositions may be prepared by known procedures using well-known and readily available ingredients.

Pharmaceutical compositions comprising cannabinol may be formulated for administration to a subject by one of a variety of standard routes, for example, ocularly, orally, topically, parenterally, by inhalation or spray, rectally or vaginally, in dosage unit formulations containing conventional non-toxic pharmaceutically acceptable carriers, adjuvants, excipients and/or vehicles.

The term parenteral as used herein includes in various embodiments subcutaneous injections, intradermal, intra-articular, intravenous, intramuscular, intravascular, intrastemal, intrathecal injection and infusion techniques. The pharmaceutical composition will typically be formulated in a format suitable for administration to the subject by the selected route, for example, as an eyedrop, an ocular ophthalmic depot, a syrup, elixir, tablet, troche, lozenge, hard or soft capsule, pill, suppository, oily or aqueous suspension, dispersible powder or granule, emulsion, injectable, or solution.

In certain embodiments, the cannabinol composition is formulated for administration via a systemic route, for example, intravenously, intramuscularly, intradermally, intraperitoneally, subcutaneously, or orally.

Compositions intended for oral use may be prepared in either solid or fluid unit dosage forms. Fluid unit dosage form can be prepared according to procedures known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. An elixir is prepared by using a hydroalcoholic (for example, ethanol) vehicle with suitable sweeteners such as sugar or saccharin, together with an aromatic flavoring agent. Suspensions can be prepared with an aqueous vehicle with the aid of a suspending agent such as acacia, tragacanth, methylcellulose and the like.

Solid formulations such as tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate: granulating and disintegrating agents for example, corn starch, or alginic acid: binding agents, for example starch, gelatin or acacia, and lubricating agents, for example magnesium stearate, stearic acid or talc and other conventional ingredients such as dicalcium phosphate, magnesium aluminum silicate, calcium sulfate, starch, lactose, methylcellulose, and functionally similar materials. The tablets may be uncoated or they may be coated by known techniques to delay disintegration and absorption in the gastrointestinal tract and thereby provide a sustained action over an extended period of time. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Soft gelatin capsules are prepared by machine encapsulation of a slurry of the compound with an acceptable vegetable oil, light liquid petrolatum or other inert oil.

Aqueous suspensions contain the active ingredient in admixture with one or more excipients suitable for the manufacture of aqueous suspensions. Such excipients include suspending agents, for example sodium carboxylmethylcellulose, methyl cellulose, hydropropylmethylcellulose, sodium alginate, polyvinylpyrrolidone, gum tragacanth and gum acacia; and dispersing or wetting agents such as naturally-occurring phosphatides (for example, lecithin), condensation products of an alkylene oxide with fatty acids (for example polyoxyethylene stearate), condensation products of ethylene oxide with long chain aliphatic alcohols (for example hepta-decaethyleneoxycetanol), condensation products of ethylene oxide with partial esters derived from fatty acids and a hexitol (for example, polyoxyethylene sorbitol monooleate), or condensation products of ethylene oxide with partial esters derived from fatty acids and hexitol anhydrides (for example polyethylene sorbitan monooleate). The aqueous suspensions may also contain one or more preservatives, for example ethyl, or n-propyl-p-hydroxy benzoate, one or more coloring agents, one or more flavoring agents or one or more sweetening agents, such as sucrose or saccharin.

Oily suspensions may be formulated by suspending the active ingredient in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oil phase may be a vegetable oil, for example olive oil or peanut oil, or a mineral oil, for example liquid paraffin, or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of such partial esters with ethylene oxide, for example polyoxyethylene sorbitan monooleate. The emulsions may also optionally contain sweetening and flavoring agents.

The pharmaceutical compositions may be in the form of a sterile injectable aqueous or oleaginous suspension. Such suspensions may be formulated as known in the art using suitable dispersing or wetting agents and suspending agents such as those mentioned above. The sterile injectable preparation may also be a sterile injectable solution or a suspension in a non-toxic parentally acceptable diluent or solvent, for example as a solution in 1,3-butanediol. Other acceptable vehicles and solvents that may be employed include, for example, water, Ringer's solution and isotonic sodium chloride solution. In addition, sterile, fixed oils may be employed as a solvent or suspending medium. Various bland fixed oils known to be suitable for this purpose may be employed including synthetic mono- or diglycerides. In addition, fatty acids such as oleic acid find use in the preparation of injectables. Adjuvants such as local anesthetics, preservatives and buffering agents may also optionally be included in the injectable solution or suspension.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, Pa. (2000).

The concentration of the neuroprotective compound (e.g., cannabinol) in the formulation will vary depending on the condition to be treated and/or the mode of administration.

### Methods

Described herein are methods of protecting a neuron from neurodegenerative stimuli. In general, the methods include contacting the neuron with an effective amount of a composition comprising cannabinol or a cannabinol derivative. The method can be an in vitro method. Alternatively, the method can be a method performed at least partially in vivo, such as by administering a neuroprotective composition to a subject. The administering can be performed by systemic (e.g., i. v., or s.c.), or localized injection. For example, localized injection can comprise ivt injection. The administering can be performed by a localized administration method that is non-invasive. For example, localized administration to retinal neurons, such as retinal ganglia can include administration of an eye drop formulation, such as a hydrogel (see, e.g., WO 2018/205022) or a microemulsion (see, e.g., U.S. Pat. No. 9,149,453).

In certain embodiments, the compound is administered for a period of less than six weeks. In certain embodiments, the compound is administered for a period of about one to four weeks. In other embodiments, such as to treat a neurodegenerative disease such as glaucoma, the compound will be administered for an extended period of time, such as for several years, or for the remaining life of the patient. The compound may be administered weekly, every other day, daily, twice per day, or three times per day.

The neuroprotective compound may be administered to treat an eye of a subject in need of treatment to protect retinal neurons (e.g., optical nerve fibers). For example, the subject may have received an insult affecting the optic nerve fibers, such as a physical injury. As another example, the subject may have glaucoma or have received a diagnosis of glaucoma. If the neuroprotective compound is administered to protect neurons, such as retinal neurons, then the neuroprotective compound can be administered at a dosage that provides a peak, median, or trough, preferably peak, neuroprotective effective concentration of the neuroprotective compound (e.g., cannabinol or a derivative thereof) in contact with the target neuron or target neuronal population. In some embodiments, the target neuron is a retinal neuron. In some embodiments, the target neuron is a peripheral neuron. In some embodiments, the target neuron is a central neuron.

In an embodiment, the neuroprotective effective concentration of the neuroprotective compound (e.g., cannabinol or a derivative or a prodrug thereof) in contact with the target neuron or target neuronal population is less than about 25 μM, less than about 20 μM, less than about 15 μM, less than about 14 μM, less than about 13 μM, less than about 12 μM, or less than about 10 μM. In an embodiment, the neuroprotective effective concentration of the neuroprotective compound (e.g., cannabinol or a derivative thereof) in contact with the target neuron or target neuronal population is from greater than about 0.15 μM to less than about 25 μM, or from greater than 0.15 μM to less than 25 μM, or from at least about 0.15 μM to less than about 25 μM, or from at least 0.15 μM to less than 25 μM, or from greater than about 0.15 μM to less than about 20 μM, or from greater than 0.15 μM to less than 20 μM, or from at least about 0.15 μM to less than about 20 μM, or from at least 0.15 μM to less than 20 μM.

In an embodiment, the neuroprotective effective concentration of the neuroprotective compound (e.g., cannabinol or a derivative thereof) in contact with the target neuron or target neuronal population is from greater than about 0.15 μM to less than about 15 μM, or from greater than 0.15 μM to less than 15 μM, or from at least about 0.15 μM to less than about 15 μM, or from at least 0.15 μM to less than 15 μM, or from greater than about 0.15 μM to less than about 12 μM, or from greater than 0.15 μM to less than 12 μM, or from at least about 0.15 μM to less than about 12 μM, or from at least 0.15 μM to less than 12 μM. In an embodiment, the neuroprotective effective concentration of the neuroprotective compound (e.g., cannabinol or a derivative thereof) is from at least about 0.5 μM to less than about 25 μM, or from at least 0.5 μM to less than 25 μM, or from at least about 0.5 μM to less than about 20 μM, or from at least 0.5 μM to less than 20 μM. In an embodiment, the neuroprotective effective concentration of the neuroprotective compound (e.g., cannabinol or a derivative thereof) is from at least about 0.5 μM to less than about 15 μM, or from at least 0.5 μM to less than 15 μM, or from at least about 0.5 μM to less than about 12 μM, or from at least 0.5 μM to less than 12 μM.

In an embodiment, the neuroprotective effective concentration of the neuroprotective compound (e.g., cannabinol or a derivative thereof) in the ocular tissues inside the eye is less than about 25 μM, less than about 20 μM, less than about 15 μM, less than about 14 μM, less than about 13 μM, less than about 12 μM, or less than about 10 μM. In an embodiment, the neuroprotective effective concentration of the neuroprotective compound (e.g., cannabinol or a derivative thereof) in the ocular tissues inside the eye is from greater than about 0.15 μM to less than about 25 μM, or from greater than 0.15 μM to less than 25 μM, or from at least about 0.15 μM to less than about 25 μM, or from at least 0.15 μM to less than 25 μM, or from greater than about 0.15 μM to less than about 20 μM, or from greater than 0.15 μM to less than 20 μM, or from at least about 0.15 μM to less than about 20 μM, or from at least 0.15 μM to less than 20 μM.

In an embodiment, the neuroprotective effective concentration of the neuroprotective compound (e.g., cannabinol or a derivative thereof) in the ocular tissues inside the eye is from greater than about 0.15 μM to less than about 15 μM, or from greater than 0.15 μM to less than 15 μM, or from at least about 0.15 μM to less than about 15 μM, or from at least 0.15 μM to less than 15 μM, or from greater than about 0.15 μM to less than about 12 μM, or from greater than 0.15 μM to less than 12 μM, or from at least about 0.15 μM to less than about 12 μM, or from at least 0.15 μM to less than 12 μM. In an embodiment, the neuroprotective effective concentration of the neuroprotective compound (e.g., cannabinol or a derivative thereof) is from at least about 0.5 μM to less than about 25 μM, or from at least 0.5 μM to less than 25 μM, or from at least about 0.5 μM to less than about 20 μM, or from at least 0.5 μM to less than 20 μM. In an embodiment, the neuroprotective effective concentration of the neuroprotective compound (e.g., cannabinol or a derivative thereof) is from at least about 0.5 μM to less than about 15 μM, or from at least 0.5 μM to less than 15 μM, or from at least about 0.5 μM to less than about 12 μM, or from at least 0.5 μM to less than 12 μM.

In an embodiment, the neuroprotective effective concentration of the neuroprotective compound (e.g., cannabinol or a derivative thereof) in the ocular tissues inside the eye is from greater than about 0.15 μM to less than about 10 μM, or from greater than 0.15 μM to less than 7.5 μM, or from at least about 0.15 μM to less than about 10 μM, or from at least 0.15 μM to less than 7.5 μM, or from greater than about 0.15 μM to about 5 μM, or from greater than 0.15 μM to 5 μM, or from at least about 0.15 μM to about 5 μM, or from at least 0.15 μM to 5 μM.

For example, the neuroprotective compound can be administered orally, intrathecally, intravenously, topically, or injected, and/or administered directly to the site of the target neuron or target neuronal population. In an embodiment, the neuroprotective effective concentration is achieved by a systemic dosage of from about 1 mg/kg to about 100 mg/kg, preferably from about 1 mg/kg to about 20 mg/kg, more preferably from about 1 mg/kg to about 15 mg/kg, yet more preferably from about 1 mg/kg to about 10 mg/kg, most preferably from about 1 mg/kg to about 13 mg/kg. The dose can be repeated, e.g., weekly, every other day, daily, or twice a day.

The neuroprotective compound can be administered intrathecally, intravenously, or injected, or administered directly into the eye such as by topical eye instillation or intravitreal injection or pump. In an embodiment, for ocular administration in an ocular indication (e.g., to treat glaucoma), the systemic dose can be from 1 mg/kg to 20 mg/kg. The dose can be repeated, e.g., weekly, every other day, daily, or twice a day. In an embodiment, for systemic administration in an ocular indication (e.g., to treat glaucoma), the systemic dose can be from 1 mg/kg to 15 mg/kg, from 1 mg/kg to 13 mg/kg, or from 1 mg/kg to 10 mg/kg. The dose can be repeated, e.g., weekly, every other day, daily, or twice a day. In an embodiment, for systemic administration in a peripheral indication (e.g., to treat peripheral neuropathy and/or peripheral nerve insult or injury), the dose can be from 1 mg/kg to 20 mg/kg, from 1 mg/kg to 15 mg/kg, from 1 mg/kg to 13 mg/kg, or from 1 mg/kg to 10 mg/kg. The dose can be repeated, e.g., weekly, every other day, daily, or twice a day. In an embodiment, for systemic administration in a central indication (e.g., to treat a central nerve insult or injury), the dose can be from 1 mg/kg to 20 mg/kg, from 1 mg/kg to 15 mg/kg, from 1 mg/kg to 13 mg/kg, or from 1 mg/kg to 10 mg/kg. The dose can be repeated, e.g., weekly, daily, or twice a day.

In an embodiment, for ocular administration in an ocular indication (e.g., to treat glaucoma), the ocular dose can be from 0.5 mg to 20 mg, from 0.5 mg to 15 mg, from 0.5 mg to 10 mg, from 1 mg to 20 mg, from 1 mg to 15 mg, from 1 mg to 10 mg, from 0.5 mg to 5 mg, or from 1 mg to 5 mg applied to the eye, such as in the form of an eye drop or eye gel. The dose can be repeated, e.g., weekly, every other day, daily, or twice a day. In an embodiment, for ocular administration in an ocular indication (e.g., to treat glaucoma), the ocular dose can be from 0.05 mg to 2 mg, from 0.05 mg to 1.5 mg, from 0.05 mg to 1 mg, from 0.1 mg to 2 mg, from 0.1 mg to 1.5 mg, from 0.1 mg to 1 mg, from 0.05 mg to 0.5 mg, or from 0.1 mg to 0.5 mg applied to the eye, such as in the form of an intravitreal injection or pump. The dose can be repeated, e.g., weekly, every other day, daily, or twice a day.

In certain embodiments, the neuroprotective compound is administered within about 0-48 hours of an insult affecting the retinal neurons. In certain embodiments, the neuroprotective compound is administered within about 2-24 hours of an insult affecting the retinal neurons. In certain embodiments, the neuroprotective compound is administered within about 3-12 hours of an insult affecting the retinal neurons. In certain embodiments, the neuroprotective compound is administered within about 3-5 hours of an insult affecting the retinal neurons.

In certain embodiments, the neuroprotective compound, or a formulation thereof, is administered to a subject having diabetic retinal neuropathy. In certain embodiments, the neuroprotective compound, or a formulation thereof, is administered to a subject having AMD. In certain embodiments, the neuroprotective compound, or a formulation thereof, is administered to a subject having retinitis pigmentosa. In certain embodiments, the neuroprotective compound, or a formulation thereof, is administered to a subject having glaucoma.

The neuroprotective compound may be administered to treat a subject in need of treatment to protect peripheral neurons. For example, the subject may have received an insult affecting one or more peripheral nerves, such as a physical injury. As another example, the subject may have a disease or condition characterized by peripheral nerve degeneration

In certain embodiments, the neuroprotective compound is administered within about 0-48 hours of an insult affecting the peripheral neurons. In certain embodiments, the neuroprotective compound is administered within about 2-24 hours of an insult affecting the peripheral neurons. In certain embodiments, the neuroprotective compound is administered within about 3-12 hours of an insult affecting the peripheral neurons. In certain embodiments, the neuroprotective compound is administered within about 3-5 hours of an insult affecting the peripheral neurons.

The neuroprotective compound may be administered to treat a subject in need of treatment to protect central neurons. For example, the subject may have received an insult affecting neurons in the central nervous system (CNS). As another example, the subject may have a disease or condition characterized by central nerve degeneration.

In certain embodiments, the neuroprotective compound is administered within about 0-48 hours of an insult affecting the CNS, such as a physical injury. In certain embodiments, the neuroprotective compound is administered within about 2-24 hours of an insult affecting the CNS. In certain embodiments, the neuroprotective compound is administered within about 3-12 hours of an insult affecting the CNS. In certain embodiments, the neuroprotective compound is administered within about 3-5 hours of an insult affecting the CNS.

The method may include or further include administering a second drug active agent simultaneously or sequentially in combination with the neuroprotective composition. In some cases, the second drug active agent is a therapeutic for treatment of glaucoma. For example, the method can include administering a drug to reduce intraocular pressure in a subject in need thereof.

### EXAMPLES

### Example 1: Protection of Neuronal Cells with Cannabinol at Atmospheric Pressure

Cell Culture and Differentiation: The mouse 661W (RGC-5) cell line was maintained in DMEM cell culture medium supplemented with 10% FBS and 1% Antibiotic-Antimycotic penicillin/streptomycin (growth medium) at 37° C. in a humidified atmosphere of 5% CO₂. To induce neuronal differentiation in 661W cells, culture media was replaced by a growth medium containing 321 nM Staurosporine (STSR), and cells were incubated for 24 hrs at 37° C. in a humidified atmosphere of 5% CO₂.

Compounds and Dosing Formulations: Selected cannabinoids: CBN, CBD, CBGA, CBDA, CBND and Δ⁹-THC were procured from Cayman and Toronto Research Chemicals. Ethanol was used as the solvent to prepare 1 and 10 mM stock solutions. Treatment concentrations for CBN were prepared at 0.015, 0.05, 0.15, 0.5, 1.5, 5, 10 and 15 μM. Treatment concentrations at 0.5, 1.5 and 5 μM for other cannabinoids were prepared directly in the control medium (DMEM+5% FBS+1% Antibiotic-Antimycotic) by using appropriate stock solution.

Evaluation of Cytotoxicity and Neuroprotection: Evaluation of cannabinoid cytotoxicity on differentiated 661W cells under atmospheric pressure was carried out using MTT (3-(4,5-dimethylthiazol-2-yl)-2,5-diphenyltetrazolium bromide) assay. The cells were seeded onto 96-well plates (4,000 cells/well) in DMEM complete medium and allowed to reach ˜70% confluence for 24 hrs. After 24 hours, cell culture medium was replaced by growth medium containing 321 nM Staurosporine (STSR) and incubated for 1 day to induce differentiation into a neuronal phenotype.

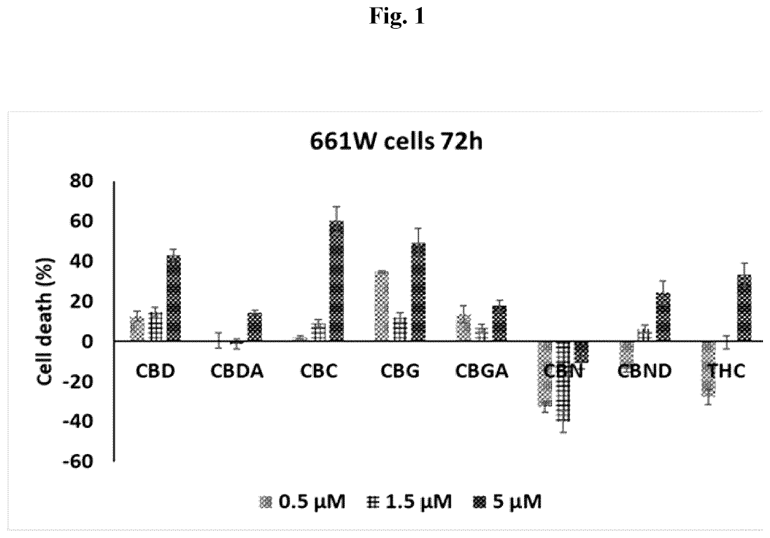

MTT Assay: For the MTT assay, differentiated 661W cells were treated with cannabinoids at various concentrations and processed to determine cytotoxicity. Briefly, 5 mg/mL of methylthiazolyldiphenyl-tetrazolium bromide (Sigma-Aldrich) stock solution was prepared in PBS. Following treatment of cannabinoids for 72 hours, 661W cells were incubated with 20 μL of MTT stock solution in 200 μL DMEM for 2 hours at 37° C. Following subsequent washes with PBS, 200 μL of isopropanol was added to each well, and the resulting change in color from dissolving formazan salt was immediately quantified using spectrophotometer (BMG Labtech) at a wavelength of 570 nm. The data was normalized to Vehicle Control (VC) containing 0.15% ethanol and presented as % Cell Death. The VC (%) at atmospheric pressure was considered as 0% cell death. The results are illustrated in FIG. 1.

### Example 2: Neuroprotection of 661W Cells with Cannabinol at Elevated Hydrostatic Pressure

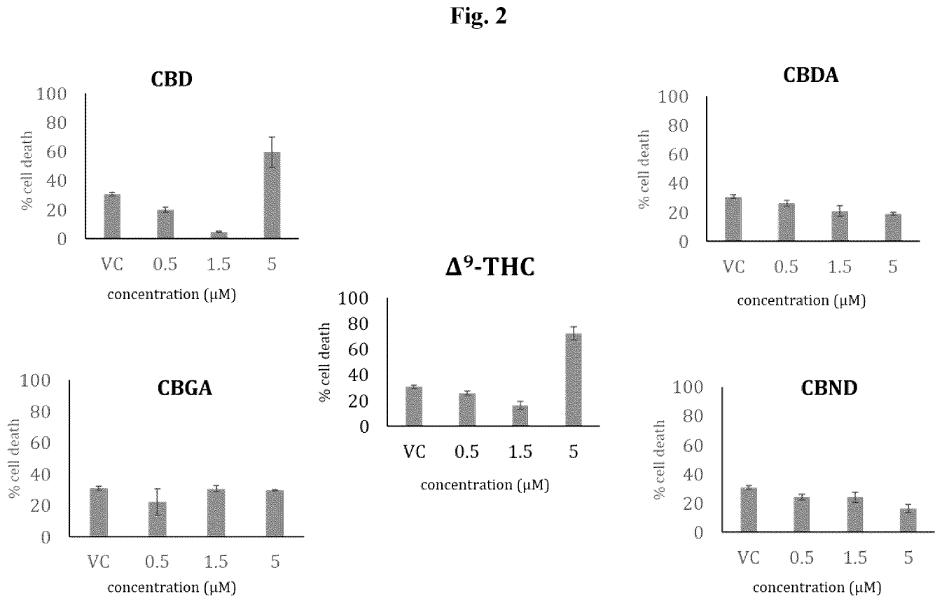

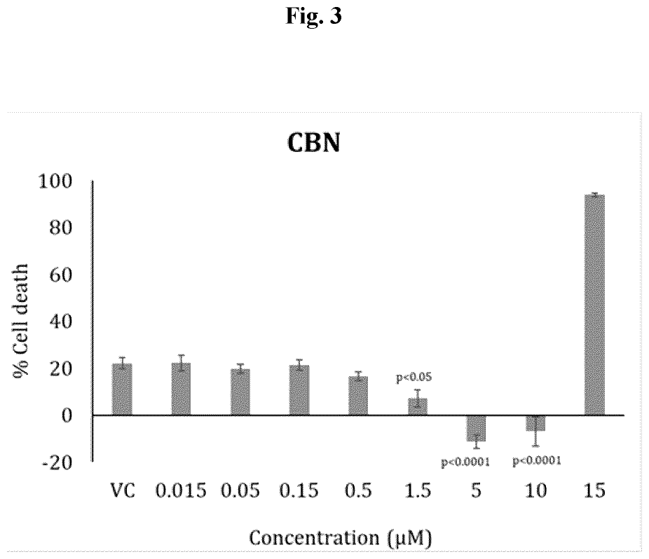

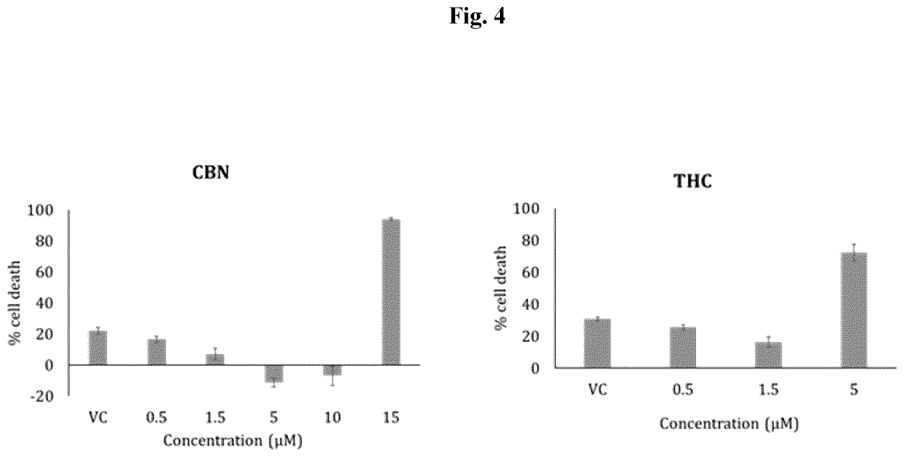

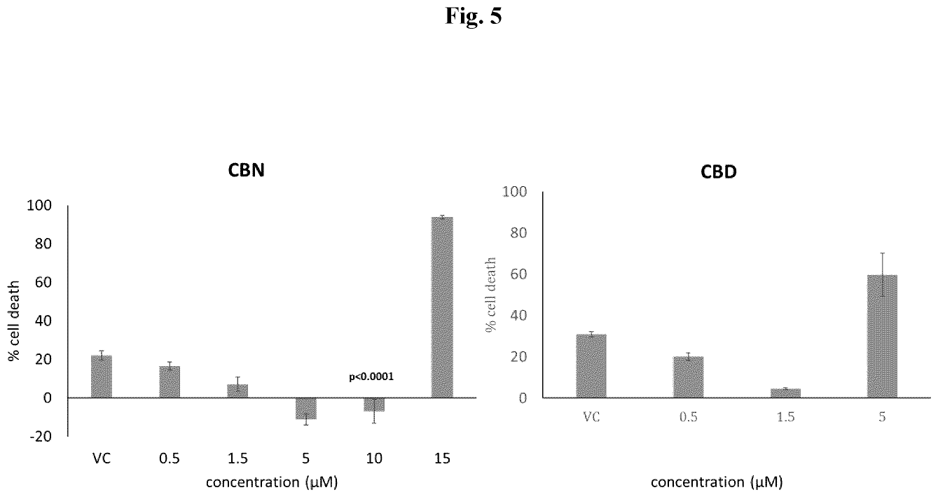

Except where indicated all procedures were performed as described in Example 1. Differentiated 661W cells treated with cannabinoids at the respective concentrations were placed in a pressurized chamber where an elevated hydrostatic pressure of 2040 mmHg was maintained for 72 hrs. At the end of incubation, 661W cells were processed for MTT assay to determine cytotoxicity. The results are illustrated in FIGS. 2-5.

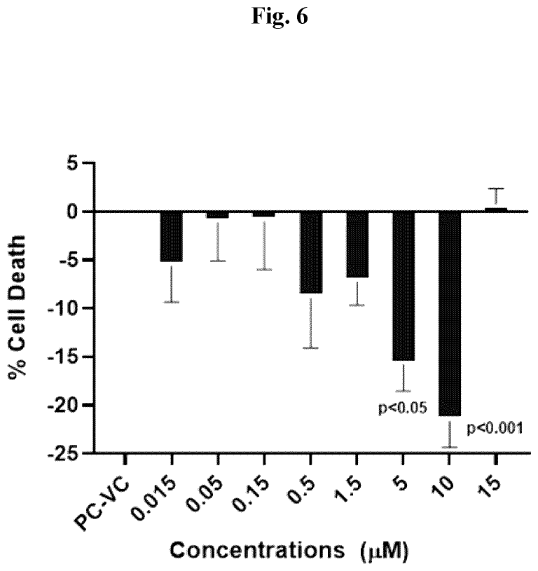

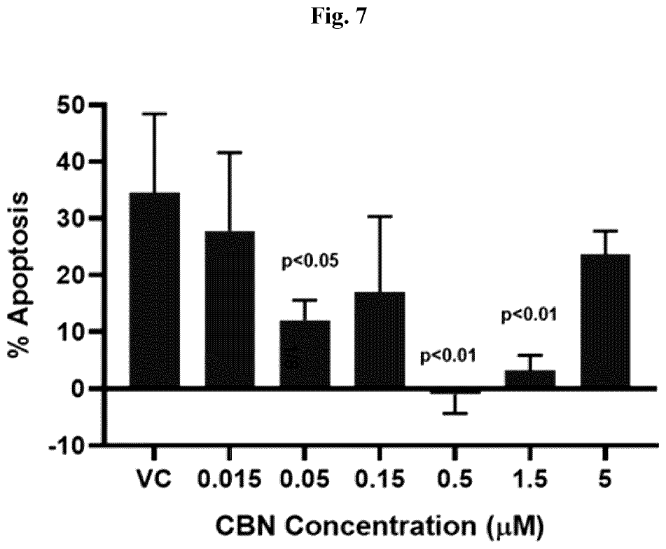

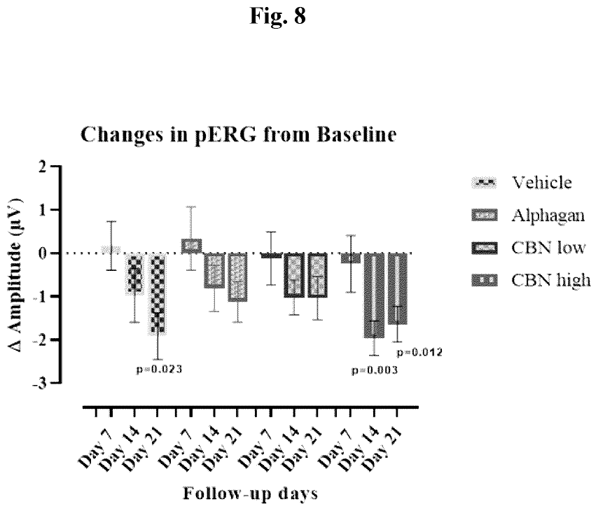

In a separate study cannabinol-derivative CBNA at 0.015, 0.05, 0.15, 0.5, 1.5, 5, 10, and 15 μM concentrations was placed on differentiated 661W cells in a pressurized chamber where an elevated hydrostatic pressure of ˜10-25 mmHg was maintained for 72 hrs. At the end of incubation, 661W cells were processed for MTT assay to determine cytotoxicity. The results are illustrated in FIG. 6.

### Example 3: Neuroprotection of 661W Cells with Cannabinol Detected Using Apoptosis Assay

Apoptosis was evaluated using Cell-APO Percentage™ apoptosis kit that detects and measures apoptosis by a colorimetric method. The assay uses Cell-APO Percentage™ apoptosis system to monitor the occurrence of apoptosis in mammalian, anchorage-dependent cells during in vitro culture. It measures the execution phase of apoptosis that has been linked to translocation of phosphatidylserine from the interior to the exterior surface of the mammalian cell membrane, experimentally supported by annexin-V binding to phosphatidylserine. Phosphatidylserine transmembrane movement results in the uptake of the APO Percentage dye by the apoptotic committed cells. This dye uptake continues until blebbing occurs and is selectively imported by cells that are undergoing apoptosis. Necrotic cells cannot retain the dye and therefore are not stained.

Differentiated 661W cells treated with Cannabinol at 0.015, 0.05, 0.15, 0.5, 1.5 and 5 μM concentrations under elevated pressure (˜20-25 mmHg) in the pressure chamber were evaluated for apoptosis according to manufacturer's instructions. Briefly, 661W cells were seeded in a 24 well tissue culture plate at 4×10⁴ cells in 500 μl culture medium and then incubated at 37° C./5% CO₂ until confluence is reached (˜24 h). Test samples of Cannabinol and vehicle control (were added to the cells at the selected concentrations and incubated for a 6 h time-period.

At 30 min before the incubation time period was reached, the original treatment medium was replaced with treatment medium containing 5% dye for all wells except a blank well and then further incubated for additional 30 min, at 37° C./5% CO₂. all medium was then removed from each well, the cells were gently washed twice with PBS (1000 μl/well) to remove non-cell bound dye. Trypsin (50 μl) was added to each well and incubated for 10 minutes at 37° C./5% CO₂. The cells were detached from the plastic, cell culture treated surface and 200 μl of dye release reagent was added to each well on a shaker plate for 10 minutes. The dye that accumulated in 30 minutes within labeled cells was released into solution and the concentration of released intracellular dye measured using a microplate colorimeter. The content of each well (250 μl ) was transferred to a 96 well flat bottom plate and read at absorbance of 550 nm, (blue-green filter), using a microplate reader. The value of the blank was subtracted from the values of all other conditions. The mean absorbance value ±standard error of the mean was plotted as a percentage of the vehicle control absorbance value.

As shown in FIG. 7, Cannabinol exhibited an effective protective effect against apoptosis when in contact with neurons under elevated pressure conditions at concentrations greater than 0.015 μM and less than 5 μM, with a statistically significant protective effect in the concentration range between 0.05 and 1.5 μM (p<0.05 and p<0.01). The elevated pressure condition in the pressurized chamber mimics the clinical situation of increased intraocular pressure in patients with glaucoma and the observed Cannabinol's suppression of apoptosis under these conditions could lead to amelioration of retinal cell degeneration and optic nerve damage.

As shown in these examples, Cannabinol exhibits a surprisingly effective neuroprotective effect when in contact with neurons at a concentration range of from about 0.15 μM to less than about 15 μM. It also demonstrated effective protection against apoptosis under elevated pressure conditions at concentration range greater than 0.015 μM and less than 5 μM. In contrast, other cannabinoids, such as CBD and THC exhibit high toxicity when in contact with neurons at a concentration of about 0.5 μM (CBD, CBG, CBGA), 1.5 μM (CBD, CBC, CBG, CBGA, CBND), or 5 μM (CBD, CBDA, CBC, CBG, CBGA, CBND, and THC). These effects are surprising in view of early publication by Colasanti et al., Exp. Eye Res. (1984) 39, 251-59, which discloses that administration of cannabinol results in neurotoxicity. The neuroprotective effect of Cannabinol was also observed under elevated pressure conditions in the pressurized chamber that mimics the clinical situation of increased intraocular pressure in glaucoma and was surprisingly superior to that of CBD and Δ⁹-THC under the same conditions. Similar neuroprotective effects were shown with the Cannabinol derivative CBNA (Formula I, wherein R¹ is H, R² is COOH, and R³ is n-C₅H₁₁.

### Example 4: Neuroprotection of Cannabinol Detected Using the Rat Episcleral Vein Laser Photocoagulation Model of Glaucoma

The pattern electroretinogram (pERG) amplitude a parameter that measures retinal ganglion cell (RGC) activity was evaluated on the eyes of anesthetized animals. During the pERG recording, the eyes were kept moist with a drop of 2.5% GonioVisc ophthalmic lubricant solution (Hub Pharmaceuticals), which also secured electrical contact between the corneas and ERG electrodes. The ERG electrodes were thin silver/silver-chloride wire rings. One eye was recorded while another eye was mechanically occluded. The occluded eye served as a reference in the ERG recording providing minimal physiological noise, while the ground electrode was placed on the tail. Only the signals from the right eye (OD) were recorded. The pERG stimulus was generated with a gamma-linearized monitor screen yielding vertical sinusoidal pattern of 0.10 cycles per degree of visual angle at viewing distance. The mean luminance of stimulation was 45 lux and contrast between dark and white was 99%. The pattern was reversed every 300 milliseconds with 1,200 reversals displayed for one recording. Two recordings were performed for each eye. For the pERG analysis, the pERG waveforms were superimposed, checked for consistency and averaged as final pERG response. Baseline pERG responses were recorded at day 0 four days prior to the lasering, and on days 7, 14 and day 21 post-lasering. Initially pERG baseline amplitudes were recorded on the eyes of all animals at baseline. The animals were then randomized into control and treatments groups based on their pERG response.

The following treatment arms were used in the study:
Group 1: Vehicle (0.5% DMSO-PBS) (IVT, day 0, day 7 and day 15) n=11
Group 2: CBN low dose, 5 μM in 0.05% DMSO-PBS (IVT, day 0, day 7 and day 15) n=13
Group 3: CBN high dose 50 μM in 0.5% DMSO-PBS (IVT, day 0, day 7 and day 15) n=11
Group 4: Brimonidine (topical, twice daily at 5 μl per eye, day −3 to day 21) n=14

FIG. 8 and Table 1 demonstrate the effect of Cannabinol on pERG activity. The functional response of RGCs measured by reduction of pERG amplitudes decreased in all treatment groups after laser treatments. However, the statistically significant difference in disease induction was observed only in the Vehicle group at day 21 ([1.92 μV] vs baseline [3.84 μV]) and in the CBN high dose group on both days 14 and 21 (day 14 [1.87 μV] and day 21 [2.19 μV] vs baseline [3.83 μV], Table 1). The pERG amplitudes in the Brimonidine (ALPHAGAN) group and in the CBN low dose group did not differ significantly from the baseline following disease induction on both of the follow-up days 14 and 21. This data indicates that CBN low dose of 5 μM final concentration inside the eye confers neuroprotective effect on RGCs similar to ALPHAGAN.

TABLE 1
pERG Amplitude Values (mean ± SEM) and Amplitude
Reduction as Compared to the Baseline Values (%)

| | | | | | COMPARED TO | | |
|---|---|---|---|---|---|---|---|
| | | | | | BASELINE VALUE | | |
| | BASELINE | DAY 7 | DAY 14 | DAY 21 | Day 7 | Day 14 | Day 21 |
| GROUP | μV | μV | μV | μV | % | % | % |
| Vehicle | 3.8 ± 0.5 | 3.9 ± 0.8 | 2.9 ± 0.5 | 1.9 ± 0.2 | 1.8 | −25.4 | −49.9* |
| CBN low | 3.3 ± 0.3 | 3.2 ± 0.5 | 2.3 ± 0.2 | 2.2 ± 0.3 | −3.7 | −31.2 | −31.6 |
| CBN high | 3.8 ± 0.4 | 3.6 ± 0.6 | 1.9 ± 0.3 | 2.2 ± 0.4 | −6.4 | −51.2** | −42.9* |
| Alphagan | 3.6 ± 0.4 | 3.9 ± 0.6 | 2.8 ± 0.4 | 2.4 ± 0.3 | 9.6 | −22.8 | −31.6 |

—pERG amplitude (μV) reduction in all treatment groups on follow-up days 7, 14 and 21.

statistically significant difference observed in the Vehicle group at day 21 vs baseline and in CBN high dose group on both day 14 and day 21 vs baseline (two-way ANOVA followed by Tukey's multiple comparison test, *p<0.05, **p<0.01)
pERG values (%) of ALPHAGAN group and the CBN low dose group at 5 μM final concentration inside the eye did not differ significantly from the baseline on both follow-up days 7, 14 and 21

Reference:
Kalesnykas G, Uusitalo H. Comparison of simultaneous readings of intraocular pressure in rabbits using Perkins handheld, Tono-Pen XL, and TonoVet tonometers. Graefes Arch Clin Exp Ophthalmol. 2007 May; 245(5):761-2.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed.

Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those skilled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

What is claimed is:

1. A method of protecting a neuron from neurodegeneration, the method comprising contacting the neuron with a composition comprising a neuroprotective compound an amount sufficient to inhibit neurodegeneration, wherein the neuroprotective compound comprises a compound of Formula I: 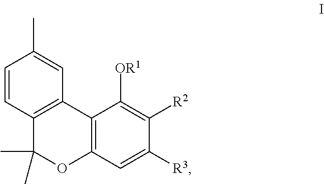
wherein
R¹ is H, R² is COOH, and R³ is n-C₅H₁₁;
10 is H, R² is H, and R³ is n-C₅H₁₁;
R¹ is CH₃, R² is H, and R³ is n-C₅H₁₁;
R¹ is H, R² is H, and R³ is n-C₄H₉;
R¹ is H, R² is H, and R³ is n-C₃H₇;
R¹ is H, R² is H, and R³ is C₂H₅; or
R¹ is H, R² is H, and R³ is CH₃,
or a derivative thereof.

2. The method of claim 1, wherein the contacting comprises administering the composition to a subject in need thereof.

3. The method of claim 1 or 2, wherein the neuron is a retinal neuron.

4. The method of claim 2 or 3, wherein the contacting comprises administering the composition to a subject in need thereof and the subject is suffering from a neurodegenerative disease.

5. The method of claim 4, wherein neurodegenerative disease is a neurodegenerative disease affecting the eye, preferably wherein the neurodegenerative disease is selected from the group consisting of glaucoma, age-related macular degeneration (AMD) retinitis pigmentosa, and diabetic retinopathy.

6. The method of claim 4, wherein neurodegenerative disease is glaucoma.

7. The method of claim 6, wherein the method comprises simultaneously or sequentially administering an additional active agent for treatment of glaucoma.

8. The method of any one of claims 1 to 7, wherein the amount sufficient to inhibit neurodegeneration is an amount sufficient to reduce an amount or rate of apoptosis of a population of neurons contacted with the composition.

9. The method of any one of claims 1 to 8, wherein the neuron is subject to elevated hydrostatic pressure and the method comprises contacting the neuron with the composition comprising the neuroprotective compound in an amount sufficient to reduce pressure-induced neurodegeneration.

10. The method of any one of claims 1 to 9, wherein the composition comprising the neuroprotective compound is provided in a microemulsion.

11. The method of any one of claims 1 to 9, wherein the composition comprising the neuroprotective compound is provided in an extended release formulation.

12. The method of claim 11, wherein the formulation comprises:
a. a delivery carrier comprising a cellulosic polymer and an anionic polysaccharide; and
b. nanoparticles comprising an amphiphilic non-ionizable block copolymer and the neuroprotective compound,
wherein the formulation has a gel point of about 30° C. to about 37° C.

13. The method of any one of claims 1 to 12, wherein the amount sufficient to inhibit neurodegeneration is an amount that results in a concentration of from about 0.15 μM to less than about 15 μM of the neuroprotective compound in contact with the neuron.

14. The method of claim 13, wherein the amount sufficient to inhibit neurodegeneration is an amount that results in a concentration of greater than about 0.5 μM and less than 15 μM of the neuroprotective compound in contact with the neuron.

15. The method of claim 14, wherein the amount sufficient to inhibit neurodegeneration is an amount that results in a concentration of from about 0.5 μM to less than 15 μM of the neuroprotective compound, preferably from greater than about 0.5 μM to less than 12 μM of the neuroprotective compound in contact with the neuron.

16. The method of claim 15, wherein the amount sufficient to inhibit neurodegeneration is an amount that results in a concentration of from about 1.5 μM to 10 μM of the neuroprotective compound in contact with the neuron.

17. The method of any one of claims 1 to 16, wherein the contacting comprises systemically administering the composition comprising the neuroprotective compound.

18. The method of claim 17, wherein the systemic administration comprises intravenous injection.

19. The method of any one of claims 1 to 16 wherein the contacting comprises local administration of the composition comprising the neuroprotective compound.

20. The method of any one of claims 1 to 16, wherein the contacting comprises administering the composition comprising the neuroprotective compound directly to the eye.

21. The method of claim 20 wherein the contacting comprises administering an eye drop formulation comprising the neuroprotective compound to the eye.

22. The method of claim 21, wherein the eye drop formulation is administered weekly, daily, or twice daily.

23. The method of any one of claims 1 to 22, wherein the neuroprotective compound is cannabinol, or cannabinolic acid, or a prodrug thereof.

24. The method of claim 23, wherein the neuroprotective compound is cannabinol.

25. Use of a composition comprising a neuroprotective compound as defined in claim 1, for treatment of neurodegeneration in a subject in need thereof, preferably in a method according to any one of claims 1 to 24.

26. A pharmaceutical composition comprising a neuroprotective compound in an eye drop formulation, wherein the neuroprotective compound comprises a compound of Formula I: 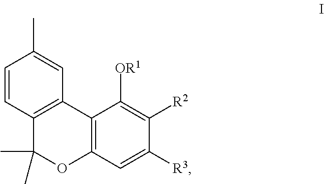
wherein
R¹ is H, R² is COOH, and R³ is n-C₅H₁₁;
10 is H, R² is H, and R³ is n-C₅H₁₁;
R¹ is CH₃, R² is H, and R³ is n-C₅H₁₁;
R¹ is H, R² is H, and R³ is n-C₄H₉;
R¹ is H, R² is H, and R³ is n-C₃H₇;
R¹ is H, R² is H, and R³ is C₂H₅; or
R¹ is H, R² is H, and R³ is CH₃,
or a derivative thereof.

27. The pharmaceutical composition of claim 26, wherein the neuroprotective compound is present at a concentration of from 0.1% w/w to 0.5% w/w.

28. The pharmaceutical composition of claim 26 or 27, wherein the neuroprotective compound is cannabinol, or cannabinolic acid.

29. The pharmaceutical composition of claim 26, 27, or 28, wherein the neuroprotective compound is cannabinol.

30. The pharmaceutical composition of any one of claims 26 to 29, wherein the neuroprotective compound is in an amount sufficient to achieve a concentration of from about 0.15 μM to less than about 15 μM of the neuroprotective compound in contact with a target neuron.

31. The pharmaceutical composition of any one of claims 26 to 30, wherein the eye drop formulation is a microemulsion, or a hydrogel formulation.

32. The pharmaceutical composition of claim 31, wherein the eye drop formulation is a a hydrogel formulation comprising: a) a delivery carrier comprising a cellulosic polymer and an anionic polysaccharide; and b) nanoparticles comprising an amphiphilic non-ionizable block copolymer and the neuroprotective compound, wherein the formulation has a gel point of about 30° C. to about 37° C.
